(19) United States
(12) Patent Application Publication
(10) Pub. No.: US 2025/0285405 A1
(43) Pub. Date: Sep. 11, 2025

(54) MULTI-MODAL BRAIN NETWORK CALCULATION METHOD, APPARATUS, DEVICE, AND STORAGE MEDIUM

(71) Applicant: SHENZHEN INSTITUTES OF ADVANCED TECHNOLOGY, SHENZHEN (CN)

(72) Inventors: SHUQIANG WANG, SHENZHEN (CN); QIANKUN ZUO, SHENZHEN (CN)

(21) Appl. No.: 19/220,118

(22) Filed: May 28, 2025

Related U.S. Application Data
(63) Continuation of application No. PCT/CN2022/134915, filed on Nov. 29, 2022.

(51) Int. Cl. G06V 10/426; G06V 10/82; G16H 50/20

(52) U.S. Cl. CPC G06V 10/426; G06V 10/82; G16H 50/20; G06V 2201/031

(57) ABSTRACT

The present disclosure discloses a multi-modal brain network calculation method, apparatus, device, and storage medium. The method is configured to train a brain disease prediction model. After the brain region structural feature and the brain region functional feature are separately extracted from magnetic resonance diffusion tensor imaging data and brain functional magnetic resonance data, a graph representation diffusion learning network is used to separate the universal feature and the unique feature in the brain region structural feature and the brain region functional feature. And then, multi-modal universal and unique feature fusion is implemented based on an alignment algorithm and adaptive weighting technology. Thus, complementary information between the multi-modal data is fully mining. The model can learn an effective feature of a related disease in a training process, and a finally obtained brain region disease prediction model has higher precision and better prediction effect.

### CROSS-REFERENCE TO RELATED APPLICATIONS

This application is a continuation of co-pending International Patent Application Number PCT/CN2022/134915, filed on Nov. 29, 2022, the disclosure of which is incorporated herein by reference in its entirety.

### TECHNICAL FIELD

The present disclosure relates to machine learning technology field, and particularly to a multi-modal brain network calculation method, apparatus, device, and storage medium.

### BACKGROUND

At present, brain diseases have become a common health problem in the world. It seriously endangers life safety of patients. Therefore, more and more attention has been paid to detection and diagnosis of the brain disease. Research direction of brain connectivity is one of these aspects. By analyzing the brain connectivity, diagnosis and pathologic traceability of neurodegenerative diseases are facilitated. Taking Alzheimer's disease (AD) as an example, the patients with AD will have brain connectivity changes during the development of the disease. These changing features may be obtained by brain images such as functional magnetic resonance imaging (fMRI) and diffusion tensor imaging (DTI). In a conventional method, a professional physician sets specific parameters, manually registers, and correcting images to obtain an effective connectivity by a software template. This traditional pathologic feature analysis method highly depends on an experience of the professional physician, has a high time cost and labor cost, and an output effect is greatly affected by parameters setting of the software template, and is not conducive to personalized accurate diagnosis and treatment.

With the development of artificial intelligence technology, there have been many brain-connected intelligent computing systems that do not need to rely on professional physicians. Predicting normal brain connectivity helps improve a diagnostic effect of neurodegenerative diseases. Herein, Alzheimer's disease (AD) is used as an example to describe a related technical background of the diagnosis of brain degenerative disease. Early AD patients may present changes in brain structural or functional connectivity that are reflected by DTI or fMRI. A current mainstream method is to set specific parameters through the software toolbox, calculate connectivity relationships between brain regions from a structural image or a functional image, construct a brain connectivity diagnosis model based on the connectivity relationships, and mine an abnormal feature related to the disease, thereby assisting early diagnosis of the neurodegenerative disease. At present, the research on the diagnosis of AD based on the brain connectivity method proposes a multi-modal method, which uses DTI and fMRI bi-modal image data to construct a connectivity edge and a node features of brain regions, and fuses topological features of the two modalities based on graph convolutional network (GCN) and uses it for the diagnosis of AD. However, the multi-modal fusion method combines universal features and unique features of each modality together, and then performs weighted fusion. This method may cause a problem of universal feature redundancy or unique feature damage, which results in a poor fusion effect.

### SUMMARY

The present disclosure provides a multi-modal brain network calculation method, apparatus, device, and storage medium, to resolve a problem of a poor prediction effect of conventional brain disease prediction method.

To resolve the foregoing problem, a technical solution used in this disclosure is that: providing a multi-modal brain network calculation method, configured to train a brain disease prediction model, wherein the brain disease prediction model comprises a feature extraction network, a graph representation diffusion learning network, a brain network reconstruction network, and a brain network boundary-aware module, the method comprising: inputting pre-acquired magnetic resonance diffusion tensor imaging data into the feature extraction network to obtain a brain region structural feature, and simultaneously preprocessing pre-acquired brain functional magnetic resonance data to obtain a brain region functional feature; by the graph representation diffusion learning network, decomposing the brain region structural feature and the brain region functional feature in topological space, to obtain a structural unique feature, a structural universal feature, a functional unique feature, and a functional universal feature; by the brain network reconstruction network, performing an alignment and an adaptive fusion on the structural unique feature, the structural universal feature, the functional unique feature, and the functional universal feature, to obtain a structural-functional brain connectivity matrix; inputting the structural-functional brain connectivity matrix into the brain network boundary-aware module for prediction, to obtain a prediction probability of having a tested disease; and reversely updating parameters of the feature extraction network, the graph representation diffusion learning network, the brain network reconstruction network, and the brain network boundary-aware module based on the predicted probability and a pre-constructed loss function.

As a further improvement of this disclosure, inputting pre-acquired magnetic resonance diffusion tensor imaging data to the feature extraction network to obtain a brain region structural feature, comprises: performing normalized coding on central point coordinates of the brain region and a relative volume of the brain region, according to anatomical brain region knowledge, to obtain a plurality of knowledge embedding vectors; inputting the magnetic resonance diffusion tensor imaging data into a convolution layer of the feature extraction network for processing, to obtain a plurality of channel vectors; and inputting the knowledge embedding vectors and the channel vectors into a Transformer network of the feature extraction network for processing, to obtain the brain region structural feature.

As a further improvement of this disclosure, the graph representation diffusion learning network comprises a discrete structure graph representation diffusion learning module, a temporal function graph representation diffusion learning module, and a spatial structure-dynamic temporal representation parsing module, the discrete structure graph representation diffusion learning module is configured to decompose the brain region structural feature in topological space, to obtain the structural unique feature and the structural universal feature, the temporal function graph representation diffusion learning module is configured to decompose the brain region functional feature in topological space, to obtain the functional unique feature and the functional universal feature, and the spatial structure-dynamic temporal representation parsing module is configured to reconstruct the structural universal feature and the structural unique feature into a new structural connectivity matrix, and reconstruct the functional universal feature and the functional unique feature into a new brain region functional feature.

As a further improvement of this disclosure, the discrete structure graph representation diffusion learning module decomposing the brain region structural feature in topological space, to obtain the structural unique feature and the structural universal feature, comprises: performing a vector inner product operation on the brain region structural feature, to obtain the structural connectivity matrix; inputting the brain region structural feature and the structural connectivity matrix into a first graph self-attention network of the discrete structure graph representation diffusion learning module; inputting an output of the first graph self-attention network into a first graph convolutional network of the discrete structure graph representation diffusion learning module, to obtain a structural universal variable and a structural unique variable; based on a reparameterization technique, sampling from the functional universal variable to obtain the functional universal feature, and sampling from the functional unique variable to obtain the functional unique feature.

The temporal function graph representation diffusion learning module decomposing the brain region functional feature in topological space to obtain the functional unique feature and the functional universal feature, comprises: performing an inter vector product operation on the brain region functional feature to obtain a functional feature matrix; inputting the brain region functional feature and the functional feature matrix into a second graph self-attention network of the discrete structure graph representation diffusion learning module; inputting an output of the second graph self-attention network into a second graph convolutional network of the temporal function graph representation diffusion learning module, to obtain a functional universal variable and a functional unique variable; and based on the reparameterization technique, sampling from functional universal variable to obtain the functional universal feature, and sampling from the functional unique variable to obtain the functional unique feature.

As a further improvement of this disclosure, the brain network reconstruction network comprises a brain network reconstruction module and a multi-modal representation distribution recognition module, the brain network reconstruction module is configured to reconstruct a structural-function brain connectivity matrix, according to the structural unique feature, the structural universal feature, the function unique feature, and the function universal feature, and the multi-modal representation distribution recognition module is configured to constraint the structural-functional brain connectivity matrix of the brain network reconstruction module by using a preset reference brain connectivity matrix as a target distribution.

As a further improvement of this disclosure, the brain network reconstruction module reconstructing a structural-functional brain connectivity matrix, according to the structural unique feature, the structural universal feature, the functional unique feature, and the functional universal feature, comprises: adding the structural universal feature and the functional universal feature with equal weights, to obtain an aligned universal feature, and splicing the aligned universal feature with the structural unique feature and the functional unique feature; performing an adaptive weighted aggregation on a spliced and aligned universal feature by a universal-unique feature fuzzy matching network layer, a spatial-time frequency precise association network layer, and a joint spatial projection normalized network layer of the brain network reconstruction module, to obtain a fusion feature; and performing an intra-vector operation on the fusion feature, and obtaining the structural-functional brain connectivity matrix by an activation function calculation.

As a further improvement of this disclosure, the loss function comprises a KL divergence and reconstruction loss function, a universal-unique comparison loss function, an adversarial loss function, and a boundary-aware loss function.

The KL divergence and reconstruction loss function is configured to guide the discrete structure graph representation diffusion learning module, the temporal function graph representation diffusion learning module, and the spatial structure-dynamic temporal representation parsing to update parameters, and is represented as:

$${L}_{KL} = {𝔼}_{S \sim {P}_{DTI}} [ KL ( {E}_{s} ( S ) \text{\vert \vert} ℕ ( 0 , 1 ) ) ] + {𝔼}_{F \sim {P}_{fMRI}} [ KL ( {E}_{f} ( F ) \text{\vert \vert} ℕ ( 0 , 1 ) ) ] ;$$

$${L}_{REC} = 𝔼 [ {‖ A + F - {D}_{sf} ( {S}_{c} , {S}_{p} , {F}_{c} , {F}_{p} ) ‖}_{2} ] ;$$

Wherein, L_{KL} represents a Kullback-Leibler divergence loss function, L_{REC} represents the reconstruction loss function, [symbol] represents an expected value, [symbol] represents a Gaussian distribution, KL represents a KL divergence, S represents the brain region structural feature, F represents the brain region functional feature, Eₛ represents the discrete structure graph representation diffusion learning module, E_{ƒ} represents the temporal function graph representation diffusion learning module, A represents the structural connectivity matrix, obtained by the vector inner product operation based on the brain region structural feature, D_{sf} represents the spatial structure-dynamic temporal representation parsing module, S_{c} represents the structural universal feature, Sₚ represents the structural unique feature, F_{c} represents the functional universal feature, and Fₚ represents the functional unique feature.

The universal-unique comparison loss function is configured to guide the discrete structure graph representation diffusion learning module and the temporal function graph representation diffusion learning module to update parameters, and is represented as:

$${L}_{Dist} = 𝔼 ( 2 - {‖ {S}_{c} - {S}_{p} ‖}_{2} - {‖ {F}_{c} - {F}_{p} ‖}_{2} ) + 𝔼 ( {‖ {S}_{c} - {F}_{c} ‖}_{2} ) ;$$

Wherein, L_{Dist} represents the universal-unique comparison loss function;

The adversarial loss function is configured to guide the brain network reconstruction module and the multi-modal representation distribution recognition module to update parameters, and is represented as:

$${L}_{D} = {𝔼}_{{A}_{p} \sim {P}_{{A}_{p}}} [ {( {D}_{c} ( {A}_{p} ) )}^{2} ] + {𝔼}_{{A}_{b} \sim {P}_{{A}_{b}}} [ {( 1 - {D}_{c} ( {A}_{b} ) )}^{2} ] ;$$

$${L}_{G} = {𝔼}_{{A}_{p} \sim {P}_{{A}_{P}}} [ {( 1 - {D}_{c} ( {A}_{p} ) )}^{2} ] ;$$

Wherein, L_{D} represents a loss function of a discriminator, L_{G} represents a loss function of a generator, P_{A}_{ₚ} represents a probability distribution of the structural-functional brain connectivity matrix, A_{b} represents the reference brain connectivity matrix, P_{A}_{_{b}} represents a probability distribution of the reference brain connectivity matrix, Aₚ represents the structural-functional brain connectivity matrix, and D_{c} represents the multi-modal representation distribution recognition module;

The boundary-aware loss function is configured to guide a brain network reconstruction module and the brain network boundary-aware module to update parameters, and is represented as:

$${L}_{L P} = {𝔼}_{{A}_{p} ∼ {P}_{{A}_{p}}} [ - I \cdot log ( C ( {A}_{p} ) ) ] + {𝔼}_{{A}_{b} ∼ {P}_{{A}_{b}}} [ - I \cdot log ( C ( {A}_{b} ) ) ] ;$$

Wherein, L_{LP} represents the boundary-aware loss function, I represents a real label vector, and C represents the brain network boundary-aware module.

To resolve the foregoing technical problem, another technical solution used in this disclosure is that: providing a multi-modal brain network calculation apparatus, comprising: a feature extraction module, configured to input pre-acquired magnetic resonance diffusion tensor imaging data into the feature extraction network to obtain a brain region structural feature, and simultaneously preprocess pre-acquired brain functional magnetic resonance data to obtain a brain region functional feature; a decomposition module, is configured to decompose the brain region structural feature and the brain region functional feature in topological space by the graph representation diffusion learning network, to obtain a structural unique feature, a structural universal feature, a functional unique feature, and a functional universal feature; a reconstruction module, configured to perform an alignment and an adaptive fusion on the structural unique feature, the structural universal feature, the functional unique feature, and the functional universal feature by the brain network reconstruction network, to obtain a structural-functional brain connectivity matrix; a prediction module, configured to input the structural-functional brain connectivity matrix into the brain network boundary-aware module for prediction, to obtain a prediction probability of having a tested disease; and an updating module, configured to reversely update parameters of the feature extraction network, the graph representation diffusion learning network, the brain network reconstruction network, and the brain network boundary-aware module based on the predicted probability and a pre-constructed loss function.

To resolve the foregoing technical problem, another technical solution used in this disclosure is that: providing a computer device, wherein the computer device comprises a processor and a memory coupled to the processor, wherein the memory stores program instructions, and when the program instructions are executed by the processor, the processor performs steps of the multi-modal brain network calculation method of any one of the foregoing.

To resolve the foregoing technical problem, another technical solution used in this disclosure is that: providing a storage medium, wherein the storage medium stores program instructions for implementing the multi-modal brain network calculation method of any one of the foregoing.

The beneficial effects of the present disclosure is that: In the multi-modal brain network calculation method in this embodiment of the present disclosure, after the brain region structural feature and the brain region functional feature are separately extracted from the magnetic resonance diffusion tensor imaging data and the brain region functional magnetic resonance data, the graph representation diffusion learning network is used to separate the universal feature and the unique feature in the brain region structural feature and the brain region functional feature. And then, multi-modal universal and unique feature fusion is implemented based on the alignment algorithm and adaptive weighting technology. Thus, complementary information between the multi-modal data is fully mining. The model can learn an effective feature of a related disease in a training process, and a finally obtained brain region disease prediction model has higher precision and better prediction effect. In addition, the brain disease prediction model end-to-end maps the image data to a brain connectivity feature, so that cumbersome image data preprocessing steps are omitted, and a degree of automation is high, thereby improving clinical diagnosis efficiency.

### BRIEF DESCRIPTION OF DRAWINGS

FIG. 1 is a block diagram of a brain disease prediction model, according to an embodiment of the present disclosure.

FIG. 2 is a flowchart of a multi-modal brain network calculation method, according to an embodiment of the present disclosure.

FIG. 3 is a block diagram of a feature extraction network, according to an embodiment of the present disclosure.

FIG. 4 is a block diagram of a discrete structure graph representation diffusion learning module, according to an embodiment of the present disclosure.

FIG. 5 is a block diagram of a spatial structure-dynamic temporal representation parsing module, according to an embodiment of the present disclosure.

FIG. 6 is a block diagram of a brain network reconstruction module, according to an embodiment of the present disclosure.

FIG. 7 is a block diagram of a multi-modal representation distribution recognition module, according to an embodiment of the present disclosure.

FIG. 8 is a block diagram of a multi-modal brain network calculation apparatus, according to an embodiment of the present disclosure.

FIG. 9 is a block diagram of a computer device, according to an embodiment of the present disclosure.

FIG. 10 is a block diagram of a storage medium, according to an embodiment of the present disclosure.

### DESCRIPTION OF EMBODIMENTS

The technical solutions in the embodiments of the present disclosure will be clearly and completely described hereinafter with reference to the accompanying drawings in the embodiments of the present application. Obviously, the described embodiments are only a part rather than all of the embodiments of the present disclosure. Based on the embodiments of the disclosure, all other embodiments obtained by a person of ordinary skill in the art without creative efforts fall within the protection scope of the present disclosure.

The terms “first”, “second”, and “third” in this application are used only for description purposes, and cannot be understood as indicating or implying relative importance or implying a quantity of indicated technical features. Therefore, a feature defined as “first”, “second”, and “third” may explicitly or implicitly include at least one feature. In the description of this application, “multiple” means at least two, for example, two or three, unless otherwise specifically limited. All directional indications (such as up, down, left, right, front, and back) in the embodiments of the present disclosure are only used to explain a relative positional relationship, a motion condition, and the like between components in a specific posture (as shown in the accompanying drawings). If the specific posture changes, the directional indication changes accordingly. In addition, the terms “include” and “have” and any variations thereof are intended to cover the inclusion of non-exclusive. For example, a process, method, system, product, or computer device that includes a series of steps or units is not limited to a listed step or unit, but optionally further includes an unlisted step or unit, or optionally further includes another step or unit inherent to the process, method, product, or computer device.

Referring to “embodiments” herein means that the specific features, structures, or features described with reference to the embodiments may be included in at least one embodiment of the present disclosure. That the phrase appears at various locations in the specification does not necessarily refer to a same embodiment, nor is it a separate or alternative embodiment mutually exclusive with another embodiment. A person skilled in the art explicitly and implicitly understands that the embodiments described in this specification may be combined with other embodiments.

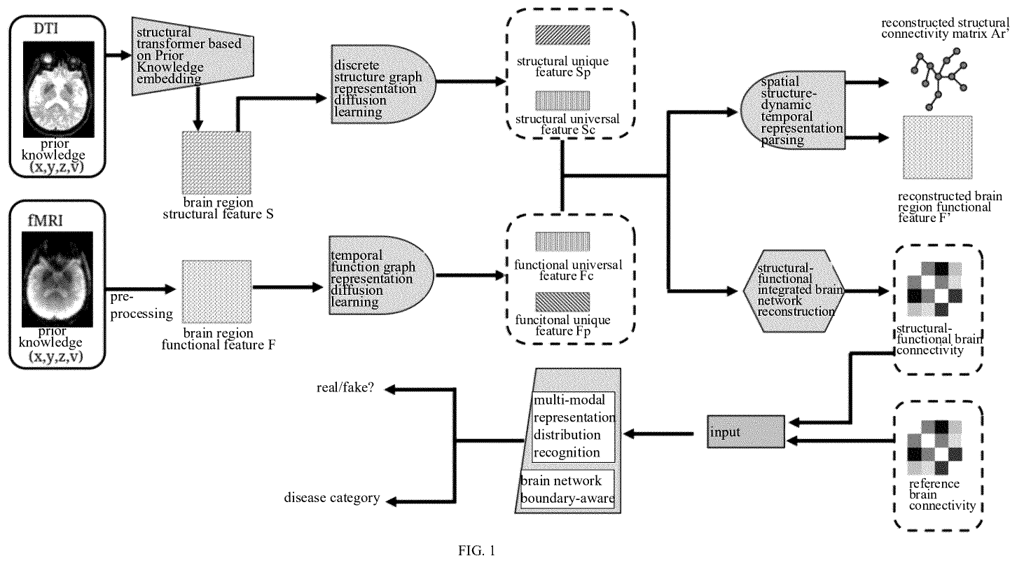

FIG. 1 is a block diagram of a brain disease prediction model, according to an embodiment of the present disclosure. As shown in FIG. 1, the brain disease prediction model includes a feature extraction network, a graph representation diffusion learning network, a brain network reconstruction network, and a brain network boundary-aware module. Wherein, the feature extraction network is a structural Transformer network based on prior knowledge embedding, and configured to learn brain region structural features from magnetic resonance diffusion tensor imaging data. The feature extraction network may guide the network to learn structural connectivity relationships between the brain regions by using prior knowledge such as central coordinates and relative volumes of the anatomical brain regions. The graph diffusion learning network is configured to decompose a brain region structural feature and a brain region functional feature in topological space, and extract a structural universal feature and a structural unique feature of a white matter fiber bundle of the brain region, and a functional universal feature and a functional unique feature of a blood oxygen time series signal of the brain region. The brain network reconstruction network is configured to align the universal and unique features of structure-function and performs an adaptive fusion thereon, improves a fusion effect of structural-functional information, and enhances a recognition capability of a brain connectivity feature. The brain network boundary-aware module enables the reconstructed structural-functional brain connectivity matrix to carry information about disease categories.

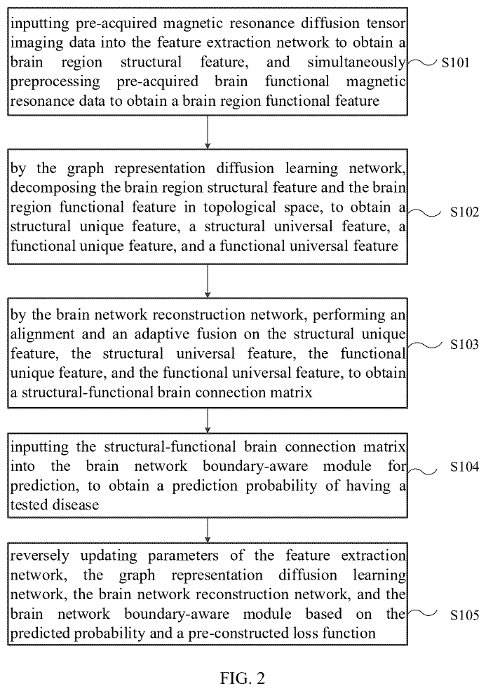

FIG. 2 is a flowchart of a multi-modal brain network calculation method, according to an embodiment of the present disclosure. It should be noted that, if there is a substantially same result, the method in the present disclosure is not limited to the procedure sequence shown in FIG. 2. As shown in FIG. 2, the multi-modal brain network calculation method includes the following steps:

S101, inputting pre-acquired magnetic resonance diffusion tensor imaging data to the feature extraction network to obtain a brain region structural feature, and simultaneously preprocessing pre-acquired brain functional magnetic resonance data to obtain a brain region functional feature.

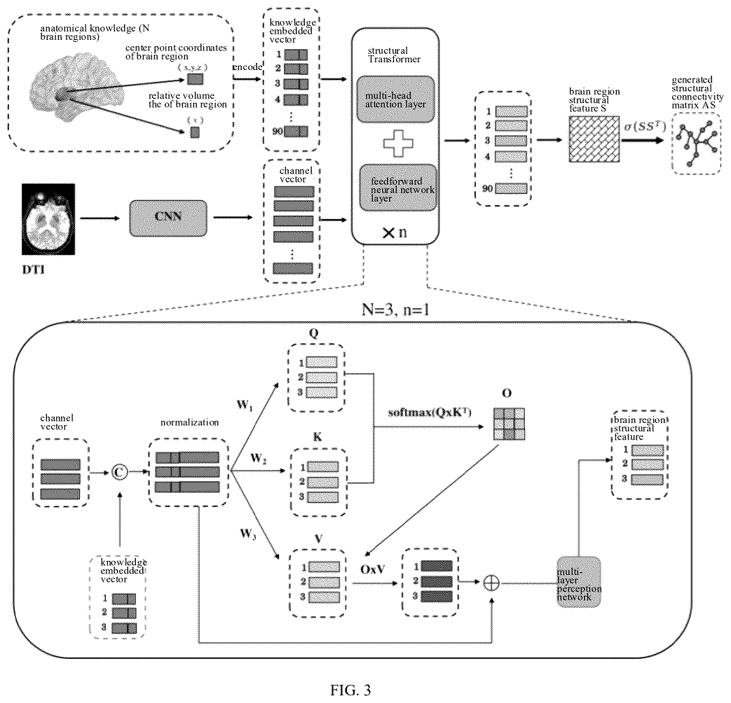

Specifically, the feature extraction network in this embodiment is a structure Transformer network based on prior knowledge embedding. Referring to FIG. 3, the Transformer network implicitly aligns a plurality of knowledge embedded vectors with a plurality of channel vectors by a parallel attention mechanism network, and maps a heterogeneous feature to subspace with similar semantics by a multi-linear normalization technology, thereby narrowing a gap in the semantics subspace, realizing a continuous dense embedding of the prior knowledge vectors in the channel vectors, improving an effect of associating the channel vectors with the brain feature, and constructing an accurate structural connectivity matrix.

Therefore, inputting pre-acquired magnetic resonance diffusion tensor imaging data into a feature extraction network to obtain a brain structure feature, specifically includes:

1. Performing normalized coding on central point coordinates of the brain region and a relative volume of the brain region, according to anatomical brain region knowledge, to obtain a plurality of knowledge embedded vectors.

2. Inputting the magnetic resonance diffusion tensor imaging data into a convolution layer of the feature extraction network for processing, to obtain a plurality of channel vectors.

Specifically, the magnetic resonance diffusion tensor imaging data is passed through an L-layer CNN convolutional layer to obtain N channel vectors, and mapped to a plurality of vectors of a dimension q by a full connectivity layer.

3. Inputting the knowledge embedded vectors and the channel vectors into the Transformer network of the feature extraction network for processing, to obtain the brain region structural feature.

S102, by the graph representation diffusion learning network, decomposing the brain region structural feature and the brain region functional feature in topological space, to obtain a structural unique feature, a structural universal feature, a functional unique feature, and a functional universal feature.

Also referring to FIG. 1, the graph representation diffusion learning network includes a discrete structure graph representation diffusion learning module, a temporal function graph representation diffusion learning module, and a spatial structure-dynamic temporal representation parsing module.

Wherein, the discrete structure graph representation diffusion learning module is configured to decompose the brain region structural feature in topological space to obtain the structural unique feature and the structural universal feature. The temporal function graph representation diffusion learning module is configured to decompose the brain region functional feature in topological space to obtain the functional unique feature and the functional universal feature.

Specifically, the central idea of the graph representation diffusion learning network is based on the graph self-attention mechanism, which decomposes the structural features or functional features of brain regions into universal parts and unique parts in the topological space, and uses the Gaussian posterior probability distribution to constrain data distribution of universal and unique features, thereby learning the features with smooth hidden layers, and improving a stability of features and a universalization performance of the model.

It should be noted that, the discrete structure graph representation diffusion learning module and the temporal function graph representation diffusion learning module have a same network structure.

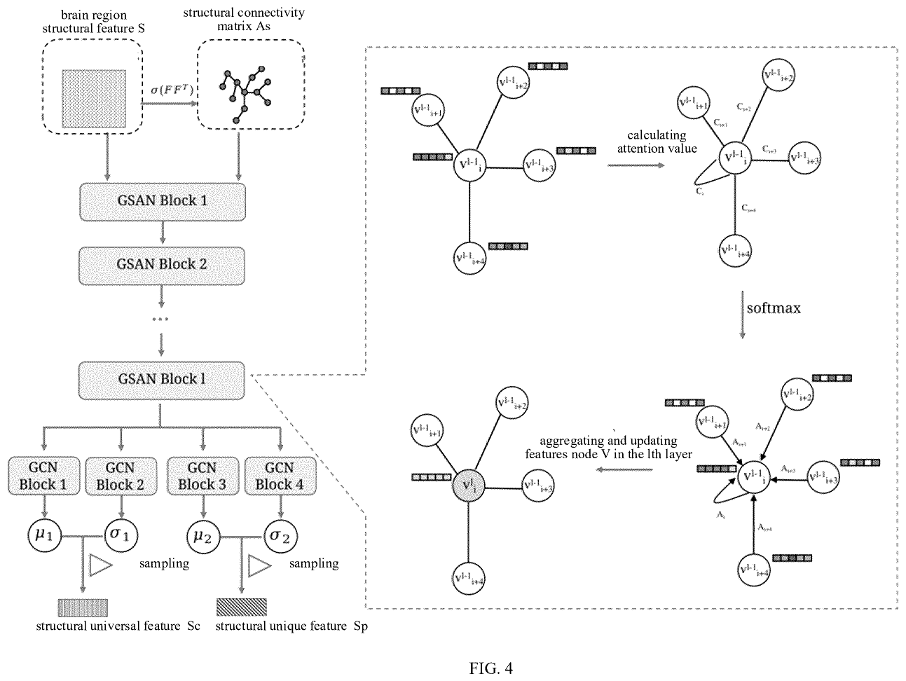

Specifically, referring to FIG. 4, the discrete structure graph representation diffusion learning module decomposing the brain region structural feature in topological space to obtain the structural unique feature and the structural universal feature, includes:

1. Performing a vector inner product operation on the brain region structural feature, to obtain a structural connectivity matrix.

2. Inputting the brain region structural feature and the structural connectivity matrix into a first graph self-attention network of the discrete structure graph representation diffusion learning module.

Specifically, a calculation process of the first graph self-attention network is as follows: at a 1-1 layer, using a node V as a center, finding nodes (Vi+1, Vi+2, Vi+3, Vi+4) connected to V, according to the structural connectivity matrix. Using features of connected nodes, linear mapping to attention values (Ci, Ci+1, Ci+2, Ci+3, Ci+4), which respectively represents a weight of a node Vi connected to nodes (Vi, Vi+1, Vi+2, Vi+3, Vi+4). Performing a Softmax calculation on the attention values to obtain a normalized attention value. Updating a feature of the node Vi according to the normalized attention value.

3. Inputting an output of the first graph self-attention network into a first graph convolutional network of the temporal function graph representation diffusion learning module, to obtain a functional universal variable and a functional unique variable.

4. Based on a reparameterization technique, sampling from the functional universal variable to obtain the functional universal feature, and sampling from the functional unique variable to obtain the functional unique feature.

It should be noted that, the first graph convolutional network includes four GCN networks, the output of the first graph self-attention network is separately input to four GCN networks, to obtain the structural universal variable (μ₁, σ₁) and the structural unique variable (μ₂, σ₂), and then by using the reparameterization technique, the structural universal feature S_{c} and the structural unique feature Sₚ can be sampled, and represented as:

$${S}_{c} = {μ}_{1} + {σ}_{1} \cdot {ε}_{1} ;$$

$${S}_{p} = {μ}_{2} + {σ}_{2} \cdot {ε}_{2} ;$$

Wherein, μ₁ and μ₂ are respectively average values of the structural universal features and the unique universal features. σ₁ and σ₂ are respectively variances of the structural universal features and the structural universal features. ε₁ and ε₂ are matrices sampled from Gaussian distribution.

Wherein, the temporal function graph representation diffusion learning module decomposing the brain region functional feature in topological space to obtain the functional unique feature and the functional universal feature, includes:

1. Performing an inter vector product operation on the brain region functional feature to obtain a functional feature matrix.

2. Inputting the brain region functional feature and the functional feature matrix into a second graph self-attention network of the discrete structure graph representation diffusion learning module.

3. Inputting an output of the second graph self-attention network into a second graph convolutional network of the temporal function graph representation diffusion learning module, to obtain a functional universal variable and a functional unique variable.

4. Based on the reparameterization technique, sampling from functional universal variable to obtain the functional universal feature, and sampling from the functional unique variable to obtain the functional unique feature.

It should be understood that, regarding the temporal function graph representation diffusion learning module and the discrete structure graph representation diffusion learning module, steps of acquiring the functional unique feature and the functional universal feature are substantially the same as steps of sampling the structural unique feature from the structural unique variable. Therefore, a process for acquiring the functional unique feature and the functional universal feature can refer to the process for sampling the structural unique feature from the structural unique variable, and detailed descriptions are omitted herein.

S103, by the brain network reconstruction network, performing an alignment and an adaptive fusion on the structural unique feature, the structural universal feature, the functional unique feature, and the functional universal feature, to obtain a structural-functional brain connectivity matrix.

In this embodiment, the spatial structure-dynamic temporal representation parsing module is configured to reconstruct the structural universal feature and the structural unique feature into a new structural connectivity matrix, and reconstruct the functional universal feature and the functional unique feature into a new brain region functional feature.

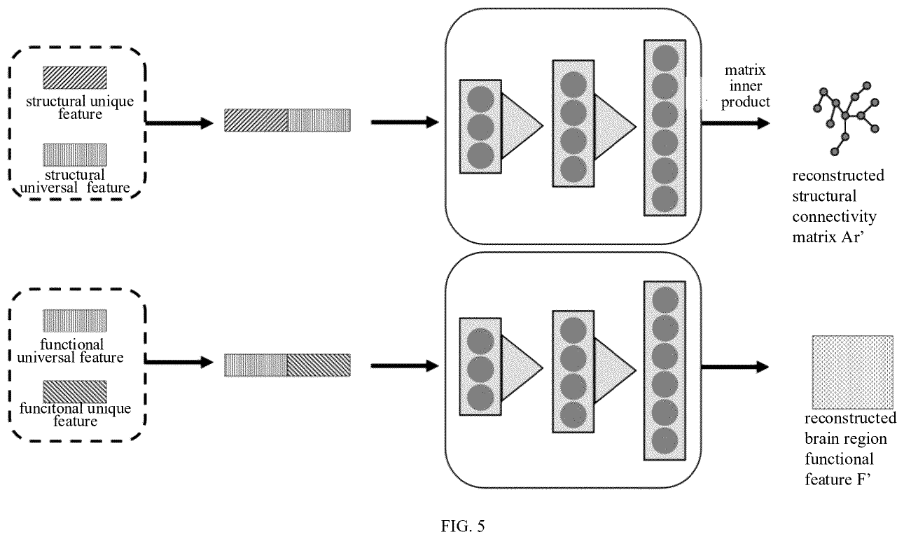

Specifically, the purpose of the time series representation parsing module is to maintain complete information of each mode and enhance a stability of graph representation diffusion learning. Referring to FIG. 5, the spatial structure-dynamic temporal representation parsing module is divided into a spatial structure parsing network and a temporal function parsing network. Two sub-networks have a same structure, but do not share parameters. Wherein, the spatial structure parsing network includes a direction projection layer, a non-linear superposition layer, and a coarse-fine grain fusion layer, and finally reconstructs the brain region structural connectivity feature. The temporal function parsing network includes a phase shift transformation layer, a similar wave packet measurement layer, and a high-low frequency aggregation layer, and finally reconstruct the brain functional feature. It should be noted that, numbers of hidden layer neurons of each layer of the spatial structure parsing network and the temporal function parsing network are respectively q′, 2q′, and q.

Further, the brain network reconstruction network includes a brain network reconstruction module and a multi-modal representation distribution recognition module. The brain network reconstruction module is configured to reconstruct a structural-functional brain connectivity matrix, according to the structural unique feature, the structural universal feature, the functional unique feature, and the functional universal feature. The multi-modal representation distribution recognition n module is configured to constraint the structural-functional brain connectivity matrix of the brain network reconstruction module by using a preset reference brain connectivity matrix as a target distribution.

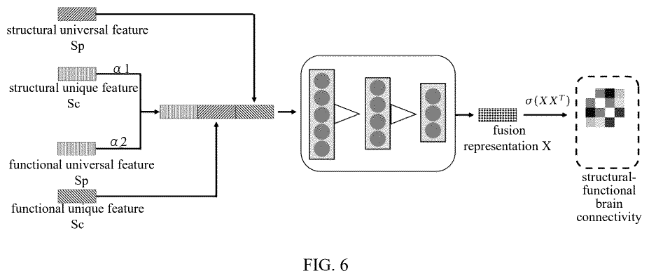

It should be noted that, mining complementary information by using structural and functional mode fusion is challenging. To resolve this problem, in the present disclosure, an adversarial learning strategy for brain network features is introduced, the universal structural and functional features are projected into a same sub-space by using an alignment technology, and the multi-modal unique feature and the aligned universal features are aggregated based on a multi-linear adaptive weighting algorithm, so as to improve a fusion effect of inter-modal complementary information. Specifically, in this embodiment, by designing the brain network reconstruction module and the multi-modal representation distribution recognition module, and using the reference brain connectivity matrix as the target distribution, a generation quality of the brain network reconstruction module is improved, inter-modal correlation and complementary information are fully fused, and a structural-functional mode fusion effect is improved.

Wherein, referring to FIG. 6, the brain network reconstruction module reconstructing a structural-function brain connectivity matrix, according to the structural unique feature, the structural universal feature, the function unique feature, and the function universal feature, includes:

1. Adding the structural universal feature and the functional universal feature with equal weights, to obtain an aligned universal feature, and splicing the aligned universal feature with the structural unique feature and the functional unique feature, which is represented as:

$${Z}_{c} = ( 0.5 * {S}_{c} + 0.5 * {F}_{c} ) ‖ {S}_{p} ‖ {F}_{p} ;$$

Wherein, ∥ represents feature splicing, Z_{c} represents a spliced and aligned universal feature, S_{c} represents the structural universal feature, Sₚ represents the structural unique feature, F_{c} represents the functional universal feature, and Fₚ represents the functional unique feature.

2. Performing an adaptive weighted aggregation on the spliced and aligned universal feature by a universal-unique feature fuzzy matching network layer, a spatial-time frequency precise association network layer, and a joint spatial projection normalized network layer of the brain network reconstruction module, to obtain a fusion feature.

It should be noted that, numbers of hidden layer neurons of the universal-unique feature fuzzy matching network layer, a spatial-time frequency precise association network layer, and a joint spatial projection normalized network layer are respectively 3q′, 2q′, and q′.

3. Performing an intra-vector operation on the fusion feature, and obtaining the structural-functional brain connectivity matrix by an activation function calculation.

Specifically, the structural-functional brain connectivity matrix is represented as follows:

Aₚ=σ(XX^{T});

Wherein, Aₚ represents the structural-functional brain connectivity matrix, σ represents the activation function, and X represents the fusion feature.

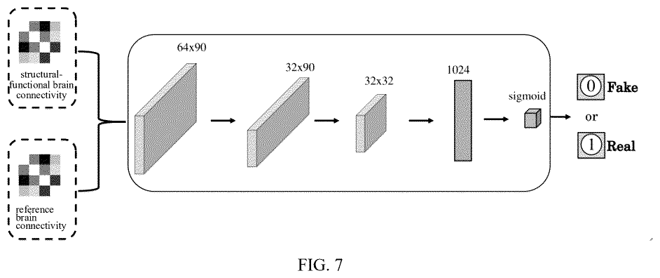

Specifically, referring to FIG. 7, the multi-modal representation distribution recognition module includes an inclination projection layer, a far field attenuation layer, a near field aggregation layer, and a coherent activation layer. A function thereof is to judge whether the reference brain connectivity matrix or the structural-functional brain connectivity matrix is real or false, and output a probability of the judgment, so as to improve the generation quality of the brain network reconstruction module, fully fuse inter-modal correlation and complementary information, and improve a fusion effect of the structural-functional mode.

S104, inputting the structural-functional brain connectivity matrix into the brain network boundary-aware module for prediction, to obtain a prediction probability of having a tested disease.

Specifically, by constraining the brain network reconstruction module, the brain network boundary-aware module makes the reconstructed structural-functional brain connectivity matrix feature to have disease category information, and output the prediction probability of the tested disease. The brain network boundary-aware module includes a brain connectivity heterogeneous propagation layer, a spatial smoothing layer, a time-frequency extension layer, and a time-space regression layer.

S105, reversely updating parameters of the feature extraction network, the graph representation diffusion learning network, the brain network reconstruction network, and the brain network boundary-aware module based on the predicted probability and a pre-constructed loss function.

Specifically, the loss function includes a KL divergence and reconstruction loss function, a universal-unique comparison loss function, an adversarial loss function, and a boundary-aware loss function.

To enable the graph representation diffusion learning module to learn the stable hidden layer feature, the universal feature and the unique feature of the brain region are constrained by using a Gaussian posterior probability distribution, and complete information of each mode is maintained by using the spatial structure-dynamic temporal representation parsing module. In this embodiment, the KL divergence and reconstruction loss function is constructed to guide the discrete structure graph representation diffusion learning module, the temporal function graph representation diffusion learning module, and the spatial structure-dynamic temporal representation parsing module to update parameters, the given brain region functional feature F˜P_{fMRI} and brain region structural feature S˜P_{DTI}, and is represented as:

$${L}_{K L} = {𝔼}_{S ∼ {P}_{DTI}} [ K L ( {E}_{s} ( S ) ‖ ℕ ( 0 , 1 ) ) ] + {E}_{F ∼ {P}_{fMRI}} [ K L ( {E}_{f} ( F ) ‖ ℕ ( 0 , 1 ) ) ] ;$$

$${L}_{R E C} = 𝔼 [ {‖ A + F - {D}_{s f} ( {S}_{c} , {S}_{p} , {F}_{c} , {F}_{p} ) ‖}_{2} ] ;$$

wherein, L_{KL} represents a Kullback-Leibler divergence loss function, L_{REC} represents a reconstruction loss function, [symbol] represents an expected value, [symbol] represents the Gaussian distribution, KL represents a KL divergence, S represents the brain region structural feature, F represents the brain region functional feature, Eₛ represents the discrete structure graph representation diffusion learning module, E_{ƒ} represents the temporal function graph representation diffusion learning module, A represents the structural connectivity matrix, obtained by the vector inner product operation based on the brain region structural feature, D_{sf} represents the spatial structure-dynamic temporal representation parsing module, S_{c} represents the structural universal feature, Sₚ represents the structural unique feature, F_{c} represents the functional universal feature, and Fₚ represents the functional unique feature.

To ensure that the universal features and unique features with complete separation are learned, and enhance the complementarity of information between modalities, a universal-unique contrast loss function is designed in this embodiment. Thus, a distance between the universal and unique features within the modalities is sufficiently far, and a distance between the universal features among the modalities is sufficiently close. The universal-unique contrast loss function is configured to guide the discrete structure graph representation diffusion learning module and the temporal function graph representation diffusion learning module to update parameters, and is represented as:

$${L}_{D i s t} = 𝔼 ( 2 - {‖ {S}_{c} - {S}_{p} ‖}_{2} - {‖ {F}_{c} - {F}_{p} ‖}_{2} ) + 𝔼 ( {‖ {S}_{c} - {F}_{c} ‖}_{2} ) ;$$

wherein, L_{Dist} represents the universal-unique comparison loss function.

To constrain the brain network reconstruction module and the multi-modal representation distribution recognition module, and learn the structural-functional brain connectivity matrix feature based on the distribution of brain connections constructed by graph convolution, the adversarial loss function is designed in this embodiment to guide the brain network reconstruction module and the multi-modal representation distribution recognition module to update parameters, and is represented as:

$${L}_{D} = {𝔼}_{{A}_{p} ∼ {P}_{{A}_{p}}} [ {( {D}_{c} ( {A}_{p} ) )}^{2} ] + {𝔼}_{{A}_{b} ∼ {P}_{{A}_{b}}} [ {( 1 - {D}_{c} ( {A}_{b} ) )}^{2} ] ;$$

$${L}_{G} = {𝔼}_{{A}_{p} ∼ {P}_{{A}_{p}}} [ {( 1 - {D}_{c} ( {A}_{p} ) )}^{2} ] ;$$

wherein, L_{D} represents a loss function of a discriminator, L_{G} represents a loss function of a generator, P_{A}_{ₚ} represents a probability distribution of the structural-functional brain connectivity matrix, A_{b} represents the reference brain connectivity matrix, P_{A}_{_{b}} represents a probability distribution of the reference brain connectivity matrix, Aₚ represents the structural-functional brain connectivity matrix, and D_{c} represents the multi-modal representation distribution recognition module;

To constrain the brain network reconstruction module, so that the fused structural-functional brain connectivity matrix has information related to the disease category, the boundary-aware loss function is designed in this embodiment to guide the brain network reconstruction module and the brain network boundary-aware module to update parameters, and is represented as:

$${L}_{L P} = {𝔼}_{{A}_{p} ∼ {P}_{{A}_{p}}} [ - I \cdot log ( C ( {A}_{p} ) ) ] + {𝔼}_{{A}_{b} ∼ {P}_{{A}_{b}}} [ - I \cdot log ( C ( {A}_{b} ) ) ] ;$$

wherein, L_{LP} represents the boundary-aware loss function, I represents a real label vector, and C represents the brain network boundary-aware module.

In the multi-modal brain network calculation method in this embodiment of the present disclosure, after the brain region structural feature and the brain region functional feature are separately extracted from the magnetic resonance diffusion tensor imaging data and the brain region functional magnetic resonance data, the graph representation diffusion learning network is used to separate the universal feature and the unique feature in the brain region structural feature and the brain region functional feature. And then, multi-modal universal and unique feature fusion is implemented based on the alignment algorithm and adaptive weighting technology. Thus, complementary information between the multi-modal data is fully mining. The model can learn an effective feature of a related disease in a training process, and a finally obtained brain region disease prediction model has higher precision and better prediction effect. In addition, the brain disease prediction model end-to-end maps the image data to a brain connectivity feature. Thus, cumbersome image data preprocessing steps are omitted, and a degree of automation is high, thereby improving clinical diagnosis efficiency.

Further, after the multi-modal brain network is trained, the brain disease prediction may be performed by the brain disease prediction model. A method for predicting the brain disease by the brain disease prediction model includes:

1. Acquiring brain functional magnetic resonance data and magnetic resonance diffusion tensor imaging data of a patient.

2. Inputting the magnetic resonance diffusion tensor imaging data to the feature extraction network to obtain a brain region structural feature, and simultaneously preprocessing the brain functional magnetic resonance data to obtain a brain region functional feature.

3. By the graph representation diffusion learning network, decomposing the brain region structural feature and the brain region functional feature in topological space, to obtain a structural unique feature, a structural universal feature, a functional unique feature, and a functional universal feature.

4. By the brain network reconstruction network, performing an alignment and an adaptive fusion on the structural unique feature, the structural universal feature, the functional unique feature, and the functional universal feature, to obtain a structural-functional brain connectivity matrix

5. Inputting the structural-functional brain connectivity matrix into the brain network boundary-aware module for prediction, to obtain a prediction probability that the patient has a corresponding disease.

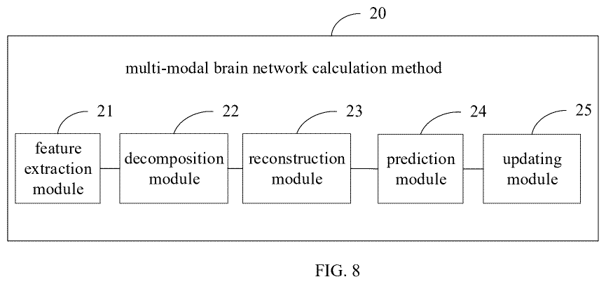

FIG. 8 is a block diagram of a multi-modal brain network calculation apparatus, according to an embodiment of the present disclosure. Referring to FIG. 8, the multi-modal brain network calculation apparatus 20 includes a feature extraction module 21, a decomposition module 22, a reconstruction module 23, a prediction module 24, and an updating module 25.

The feature extraction module 21, is configured to input pre-acquired magnetic resonance diffusion tensor imaging data into the feature extraction network to obtain a brain region structural feature, and simultaneously preprocess pre-acquired brain functional magnetic resonance data to obtain a brain region functional feature.

The decomposition module 22, is configured to decompose the brain region structural feature and the brain region functional feature in topological space by the graph representation diffusion learning network, to obtain a structural unique feature, a structural universal feature, a functional unique feature, and a functional universal feature.

The reconstruction module 23, is configured to perform an alignment and an adaptive fusion on the structural unique feature, the structural universal feature, the functional unique feature, and the functional universal feature by the brain network reconstruction network, to obtain a structural-functional brain connectivity matrix.

The prediction module 24, is configured to input the structural-functional brain connectivity matrix into the brain network boundary-aware module for prediction, to obtain a prediction probability of having a tested disease.

The updating module 25, is configured to reversely update parameters of the feature extraction network, the graph representation diffusion learning network, the brain network reconstruction network, and the brain network boundary-aware module based on the predicted probability and a pre-constructed loss function.

Optionally, the feature extraction module 21 performs to input pre-acquired magnetic resonance diffusion tensor imaging data to the feature extraction network to obtain a brain region structural feature, includes: performing normalized coding on central point coordinates of the brain region and a relative volume of the brain region, according to anatomical brain region knowledge, to obtain a plurality of knowledge embedding vectors; inputting the magnetic resonance diffusion tensor imaging data into a convolution layer of the feature extraction network for processing, to obtain a plurality of channel vectors; and inputting the knowledge embedding vectors and the channel vectors into a Transformer network of the feature extraction network for processing, to obtain the brain region structural feature.

Optionally, the graph representation diffusion learning network includes a discrete structure graph representation diffusion learning module, a temporal function graph representation diffusion learning module, and a spatial structure-dynamic temporal representation parsing module, the discrete structure graph representation diffusion learning module is configured to decompose the brain region structural feature in topological space, to obtain the structural unique feature and the structural universal feature, the temporal function graph representation diffusion learning module is configured to decompose the brain region functional feature in topological space, to obtain the functional unique feature and the functional universal feature, and the spatial structure-dynamic temporal representation parsing module is configured to reconstruct the structural universal feature and the structural unique feature into a new structural connectivity matrix, and reconstruct the functional universal feature and the functional unique feature into a new brain region functional feature.

Optionally, the discrete structure graph representation diffusion learning module decomposes the brain region structural feature in topological space, to obtain the structural unique feature and the structural universal feature, includes: performing a vector inner product operation on the brain region structural feature, to obtain the structural connectivity matrix; inputting the brain region structural feature and the structural connectivity matrix into a first graph self-attention network of the discrete structure graph representation diffusion learning module; inputting an output of the first graph self-attention network into a first graph convolutional network of the discrete structure graph representation diffusion learning module, to obtain a structural universal variable and a structural unique variable; and based on a reparameterization technique, sampling from the functional universal variable to obtain the functional universal feature, and sampling from the functional unique variable to obtain the functional unique feature.

Optionally, the temporal function graph representation diffusion learning module decomposing the brain region functional feature in topological space to obtain the functional unique feature and the functional universal feature, includes: performing an inter vector product operation on the brain region functional feature to obtain a functional feature matrix; inputting the brain region functional feature and the functional feature matrix into a second graph self-attention network of the discrete structure graph representation diffusion learning module; inputting an output of the second graph self-attention network into a second graph convolutional network of the temporal function graph representation diffusion learning module, to obtain a functional universal variable and a functional unique variable; and based on based on the reparameterization technique, sampling from functional universal variable to obtain the functional universal feature, and sampling from the functional unique variable to obtain the functional unique feature.

Optionally, the brain network reconstruction network includes a brain network reconstruction module and a multi-modal representation distribution recognition module, the brain network reconstruction module is configured to reconstruct a structural-function brain connectivity matrix, according to the structural unique feature, the structural universal feature, the function unique feature, and the function universal feature, and the multi-modal representation distribution recognition module is configured to constraint the structural-functional brain connectivity matrix of the brain network reconstruction module by using a preset reference brain connectivity matrix as a target distribution.

Optionally, the brain network reconstruction module reconstructing a structural-function brain connectivity matrix, according to the structural unique feature, the structural universal feature, the function unique feature, and the function universal feature, includes: adding the structural universal feature and the functional universal feature with equal weights, to obtain an aligned universal feature, and splicing the aligned universal feature with the structural unique feature and the functional unique feature; performing an adaptive weighted aggregation on a spliced and aligned universal feature by a universal-unique feature fuzzy matching network layer, a spatial-time frequency precise association network layer, and a joint spatial projection normalized network layer of the brain network reconstruction module, to obtain a fusion feature; and performing an intra-vector operation on the fusion feature, and obtaining the structural-functional brain connectivity matrix by an activation function calculation

Optionally, the loss function includes a KL divergence and reconstruction loss function, a universal-unique comparison loss function, an adversarial loss function, and a boundary-aware loss function.

The KL divergence and reconstruction loss function is configured to guide the discrete structure graph representation diffusion learning module, the temporal function graph representation diffusion learning module, and the spatial structure-dynamic temporal representation parsing to update parameters, and is represented as:

$${L}_{K L} = {𝔼}_{S ∼ {P}_{DTI}} [ K L ( {E}_{s} ( S ) ‖ ℕ ( 0 , 1 ) ) ] + {E}_{F ∼ {P}_{fMRI}} [ K L ( {E}_{f} ( F ) ‖ ℕ ( 0 , 1 ) ) ] ;$$

$${L}_{R E C} = 𝔼 [ {‖ A + F - {D}_{s f} ( {S}_{c} , {S}_{p} , {F}_{c} , {F}_{p} ) ‖}_{2} ] ;$$

wherein, L_{KL} represents a Kullback-Leibler divergence loss function, L_{REC} represents a reconstruction loss function, [symbol] represents an expected value, [symbol] represents a Gaussian distribution, KL represents a KL divergence, S represents the brain region structural feature, F represents the brain region functional feature, Eₛ represents the discrete structure graph representation diffusion learning module, E_{ƒ} represents the temporal function graph representation diffusion learning module, A represents the structural connectivity matrix, obtained by the vector inner product operation based on the brain region structural feature, D_{sf} represents the spatial structure-dynamic temporal representation parsing module, S_{c} represents the structural universal feature, Sₚ represents the structural unique feature, F_{c} represents the functional universal feature, and Fₚ represents the functional unique feature;

The universal-unique comparison loss function is configured to guide the discrete structure graph representation diffusion learning module and the temporal function graph representation diffusion learning module to update parameters, and is represented as:

$${L}_{D i s t} = 𝔼 ( 2 - {‖ {S}_{c} - {S}_{p} ‖}_{2} - {‖ {F}_{c} - {F}_{p} ‖}_{2} ) + 𝔼 ( {‖ {S}_{c} - {F}_{c} ‖}_{2} ) ;$$

Wherein, L_{Dist} represents the universal-unique comparison loss function;

The adversarial loss function is configured to guide the brain network reconstruction module and the multi-modal representation distribution recognition module to update parameters, and is represented as:

$${L}_{D} = {𝔼}_{{A}_{p} ∼ {P}_{{A}_{p}}} [ {( {D}_{c} ( {A}_{p} ) )}^{2} ] + {𝔼}_{{A}_{b} ∼ {P}_{{A}_{b}}} [ {( 1 - {D}_{c} ( {A}_{b} ) )}^{2} ]$$

$${L}_{G} = {𝔼}_{{A}_{p} ∼ {P}_{{A}_{p}}} [ {( 1 - {D}_{c} ( {A}_{p} ) )}^{2} ] ;$$

Wherein, L_{D} represents a loss function of a discriminator, L_{G} represents a loss function of a generator, P_{A}_{ₚ} represents a probability distribution of the structural-functional brain connectivity matrix, A_{b} represents the reference brain connectivity matrix, P_{A}_{_{b}} represents a probability distribution of the reference brain connectivity matrix, Aₚ represents the structural-functional brain connectivity matrix, and D_{c} represents the multi-modal representation distribution recognition module;

The boundary-aware loss function is configured to guide a brain network reconstruction module and the brain network boundary-aware module to update parameters, and is represented as:

$${L}_{L P} = {𝔼}_{{A}_{p} ∼ {P}_{{A}_{p}}} [ - I \cdot log ( C ( {A}_{p} ) ) ] + {𝔼}_{{A}_{b} ∼ {P}_{{A}_{b}}} [ - I \cdot log ( C ( {A}_{b} ) ) ] ;$$

Wherein, L_{LP} represents the boundary-aware loss function, I represents a real label vector, and C represents the brain network boundary-aware module.

For other details of the technical solutions of the modules in the above-mentioned multi-modal brain network calculation apparatus, refer to descriptions in the multi-modal brain network calculation method in the above-mentioned embodiment. Details are omitted herein.

It should be noted that each embodiment in this specification is described in a progressive manner. Each embodiment focuses on a difference from another embodiment. For a same similar part of the embodiments, refer to each other. For an apparatus embodiment, because the apparatus embodiment is basically similar to the method embodiment, description is relatively simple. For related parts, refer to partial descriptions of the method embodiment.

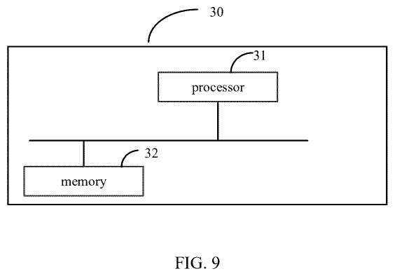

Referring to FIG. 9, FIG. 9 is a schematic structural diagram of a computer device according to an embodiment of the present invention. As shown in FIG. 9, the computer device 30 includes a processor 31 and a memory 32 coupled to the processor 31. The memory 32 stores program instructions. When the program instructions are executed by the processor 31, the processor 31 performs the steps of the multi-modal brain network calculation method described in any one of the foregoing embodiments.

The processor 31 may also be referred to as a CPU (Central Processing Unit, Central Processing Unit). The processor 52 may be an integrated circuit chip, and have a signal processing capability. The processor 52 may further be a universal-purpose processor, a digital signal processor (DSP), an application-specific integrated circuit (ASIC), a field programmable gate array (FPGA) or another programmable logic device, a discrete gate or transistor logic device, or a discrete hardware component. The universal-purpose processor may be a microprocessor, or the processor may be any conventional processor or the like.

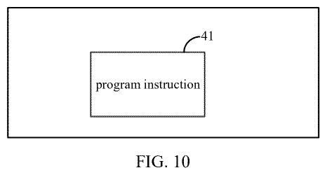

Referring to FIG. 10, FIG. 10 is a block diagram of a storage medium, according to an embodiment of the present disclosure. The storage medium in this embodiment of the present invention stores program instructions 41 that can implement the foregoing multi-modal brain network calculation method. The program instructions 41 may be stored in the foregoing storage medium in a form of a software product, and includes several instructions for instructing a computer device (which may be a personal computer, a server, or a network device) or a processor (processor) to perform all or a part of the steps of the methods in the implementation manners of this disclosure. The foregoing storage medium includes any medium that can store program code, such as a USB flash drive, a removable hard disk, a read-only memory (ROM, Read-Only Memory), a random-access memory (RAM, Random Access Memory), a magnetic disk, or an optical disc, or a computer device such as a computer, a server, a mobile phone, or a tablet.

In the embodiments provided in the present disclosure, it should be understood that the disclosed system, apparatus, and method may be implemented in another manner. For example, the described system embodiment is merely an example. For example, unit division is merely logical function division. In actual implementation, there may be another division manner. For example, multiple units or components may be combined or integrated into another system, or some features may be ignored or not performed. On the other hand, the displayed or discussed mutual coupling or direct coupling or communication connectivity may be through some interfaces, indirect coupling or communication connectivity of the apparatus or unit, and may be in an electrical, mechanical, or other form.

In addition, functional units in the embodiments of the present disclosure may be integrated into one processing unit, or each unit may exist separately physically, or two or more units may be integrated into one unit. The foregoing integrated unit may be implemented in a form of hardware, or may be implemented in a form of a software functional unit. The foregoing descriptions are merely implementations of the present disclosure, and are not intended to limit the scope of the present disclosure. Any equivalent structure or equivalent procedure transformation performed by using the content in the specification and accompanying drawings of the present disclosure, or directly or indirectly applied in another related technical field is included in the protection scope of the present disclosure.

What is claimed is:

1. A multi-modal brain network calculation method, configured to train a brain disease prediction model, wherein the brain disease prediction model comprises a feature extraction network, a graph representation diffusion learning network, a brain network reconstruction network, and a brain network boundary-aware module, the method comprising:
inputting pre-acquired magnetic resonance diffusion tensor imaging data into the feature extraction network to obtain a brain region structural feature, and simultaneously preprocessing pre-acquired brain functional magnetic resonance data to obtain a brain region functional feature;
by the graph representation diffusion learning network, decomposing the brain region structural feature and the brain region functional feature in topological space, to obtain a structural unique feature, a structural universal feature, a functional unique feature, and a functional universal feature;
by the brain network reconstruction network, performing an alignment and an adaptive fusion on the structural unique feature, the structural universal feature, the functional unique feature, and the functional universal feature, to obtain a structural-functional brain connectivity matrix;
inputting the structural-functional brain connectivity matrix into the brain network boundary-aware module for prediction, to obtain a prediction probability of having a tested disease; and
reversely updating parameters of the feature extraction network, the graph representation diffusion learning network, the brain network reconstruction network, and the brain network boundary-aware module based on the predicted probability and a pre-constructed loss function.

2. The multi-modal brain network calculation method according to claim 1, wherein inputting pre-acquired magnetic resonance diffusion tensor imaging data to the feature extraction network to obtain a brain region structural feature, comprises:
performing normalized coding on central point coordinates of the brain region and a relative volume of the brain region, according to anatomical brain region knowledge, to obtain a plurality of knowledge embedding vectors;
inputting the magnetic resonance diffusion tensor imaging data into a convolution layer of the feature extraction network for processing, to obtain a plurality of channel vectors; and
inputting the knowledge embedding vectors and the channel vectors into a Transformer network of the feature extraction network for processing, to obtain the brain region structural feature.

3. The multi-modal brain network calculation method according to claim 1, wherein the graph representation diffusion learning network comprises a discrete structure graph representation diffusion learning module, a temporal function graph representation diffusion learning module, and a spatial structure-dynamic temporal representation parsing module, the discrete structure graph representation diffusion learning module is configured to decompose the brain region structural feature in topological space, to obtain the structural unique feature and the structural universal feature, the temporal function graph representation diffusion learning module is configured to decompose the brain region functional feature in topological space, to obtain the functional unique feature and the functional universal feature, and the spatial structure-dynamic temporal representation parsing module is configured to reconstruct the structural universal feature and the structural unique feature into a new structural connectivity matrix, and reconstruct the functional universal feature and the functional unique feature into a new brain region functional feature.

4. The multi-modal brain network calculation method according to claim 3, wherein the discrete structure graph representation diffusion learning module decomposing the brain region structural feature in topological space, to obtain the structural unique feature and the structural universal feature, comprises:
performing a vector inner product operation on the brain region structural feature, to obtain the structural connectivity matrix;
inputting the brain region structural feature and the structural connectivity matrix into a first graph self-attention network of the discrete structure graph representation diffusion learning module;
inputting an output of the first graph self-attention network into a first graph convolutional network of the discrete structure graph representation diffusion learning module, to obtain a structural universal variable and a structural unique variable;
based on a reparameterization technique, sampling from the functional universal variable to obtain the functional universal feature, and sampling from the functional unique variable to obtain the functional unique feature;
the temporal function graph representation diffusion learning module decomposing the brain region functional feature in topological space to obtain the functional unique feature and the functional universal feature, comprises:
performing an inter vector product operation on the brain region functional feature to obtain a functional feature matrix;
inputting the brain region functional feature and the functional feature matrix into a second graph self-attention network of the discrete structure graph representation diffusion learning module;
inputting an output of the second graph self-attention network into a second graph convolutional network of the temporal function graph representation diffusion learning module, to obtain a functional universal variable and a functional unique variable; and
based on the reparameterization technique, sampling from functional universal variable to obtain the functional universal feature, and sampling from the functional unique variable to obtain the functional unique feature.

5. The multi-modal brain network calculation method according to claim 3, wherein the brain network reconstruction network comprises a brain network reconstruction module and a multi-modal representation distribution recognition module, the brain network reconstruction module is configured to reconstruct a structural-functional brain connectivity matrix, according to the structural unique feature, the structural universal feature, the functional unique feature, and the functional universal feature, and the multi-modal representation distribution recognition module is configured to constraint the structural-functional brain connectivity matrix of the brain network reconstruction module by using a preset reference brain connectivity matrix as a target distribution.

6. The multi-modal brain network calculation method according to claim 5, wherein the brain network reconstruction module reconstructing a structural-functional brain connectivity matrix, according to the structural unique feature, the structural universal feature, the functional unique feature, and the functional universal feature, comprises:
adding the structural universal feature and the functional universal feature with equal weights, to obtain an aligned universal feature, and splicing the aligned universal feature with the structural unique feature and the functional unique feature;
performing an adaptive weighted aggregation on a spliced and aligned universal feature by a universal-unique feature fuzzy matching network layer, a spatial-time frequency precise association network layer, and a joint spatial projection normalized network layer of the brain network reconstruction module, to obtain a fusion feature; and
performing an intra-vector operation on the fusion feature, and obtaining the structural-functional brain connectivity matrix by an activation function calculation.

7. The multi-modal brain network calculation method according to claim 6, wherein the loss function comprises a KL divergence and reconstruction loss function, a universal-unique comparison loss function, an adversarial loss function, and a boundary-aware loss function;
the KL divergence and reconstruction loss function is configured to guide the discrete structure graph representation diffusion learning module, the temporal function graph representation diffusion learning module, and the spatial structure-dynamic temporal representation parsing to update parameters, and is represented as:
$${L}_{K L} = {𝔼}_{S ∼ {P}_{DTI}} [ K L ( {E}_{s} ( S ) ‖ ℕ ( 0 , 1 ) ) ] + {E}_{F ∼ {P}_{fMRI}} [ K L ( {E}_{f} ( F ) ‖ ℕ ( 0 , 1 ) ) ] ;$$
$${L}_{R E C} = 𝔼 [ {‖ A + F - {D}_{s f} ( {S}_{c} , {S}_{p} , {F}_{c} , {F}_{p} ) ‖}_{2} ] ;$$
wherein, L_{KL} represents a Kullback-Leibler divergence loss function, L_{REC} represents a reconstruction loss function, [symbol] represents an expected value, [symbol] represents a Gaussian distribution, KL represents a KL divergence, S represents the brain region structural feature, F represents the brain region functional feature, Eₛ represents the discrete structure graph representation diffusion learning module, E_{ƒ} represents the temporal function graph representation diffusion learning module, A represents the structural connectivity matrix, obtained by the vector inner product operation based on the brain region structural feature, D_{sf} represents the spatial structure-dynamic temporal representation parsing module, S_{c} represents the structural universal feature, Sₚ represents the structural unique feature, F_{c} represents the functional universal feature, and Fₚ represents the functional unique feature;
the universal-unique comparison loss function is configured to guide the discrete structure graph representation diffusion learning module and the temporal function graph representation diffusion learning module to update parameters, and is represented as:
$${L}_{D i s t} = 𝔼 ( 2 - {‖ {S}_{c} - {S}_{p} ‖}_{2} - {‖ {F}_{c} - {F}_{p} ‖}_{2} ) + 𝔼 ( {‖ {S}_{c} - {F}_{c} ‖}_{2} ) ;$$
wherein, L_{Dist} represents the universal-unique comparison loss function;
the adversarial loss function is configured to guide the brain network reconstruction module and the multi-modal representation distribution recognition module to update parameters, and is represented as:
$${L}_{D} = {𝔼}_{{A}_{p} ∼ {P}_{{A}_{p}}} [ {( {D}_{c} ( {A}_{p} ) )}^{2} ] + {𝔼}_{{A}_{b} ∼ {P}_{{A}_{b}}} [ {( 1 - {D}_{c} ( {A}_{b} ) )}^{2} ] ;$$
$${L}_{G} = {𝔼}_{{A}_{p} ∼ {P}_{{A}_{p}}} [ {( 1 - {D}_{c} ( {A}_{p} ) )}^{2} ] ;$$
wherein, L_{D} represents a loss function of a discriminator, L_{G} represents a loss function of a generator, P_{A}_{ₚ} represents a probability distribution of the structural-functional brain connectivity matrix, A_{b} represents the reference brain connectivity matrix, P_{A}_{_{b}} represents a probability distribution of the reference brain connectivity matrix, Aₚ represents the structural-functional brain connectivity matrix, and D_{c} represents the multi-modal representation distribution recognition module;
the boundary-aware loss function is configured to guide the brain network reconstruction module and the brain network boundary-aware module to update parameters, and is represented as:
$${L}_{L P} = {𝔼}_{{A}_{p} ∼ {P}_{{A}_{p}}} [ - I \cdot log ( C ( {A}_{p} ) ) ] + {𝔼}_{{A}_{b} ∼ {P}_{{A}_{b}}} [ - I \cdot log ( C ( {A}_{b} ) ) ] ;$$
wherein, L_{LP} represents the boundary-aware loss function, I represents a real label vector, and C represents the brain network boundary-aware module.

8. A multi-modal brain network calculation apparatus, comprising:
a feature extraction module, configured to input pre-acquired magnetic resonance diffusion tensor imaging data into the feature extraction network to obtain a brain region structural feature, and simultaneously preprocess pre-acquired brain functional magnetic resonance data to obtain a brain region functional feature;
a decomposition module, is configured to decompose the brain region structural feature and the brain region functional feature in topological space by the graph representation diffusion learning network, to obtain a structural unique feature, a structural universal feature, a functional unique feature, and a functional universal feature;
a reconstruction module, configured to perform an alignment and an adaptive fusion on the structural unique feature, the structural universal feature, the functional unique feature, and the functional universal feature by the brain network reconstruction network, to obtain a structural-functional brain connectivity matrix;
a prediction module, configured to input the structural-functional brain connectivity matrix into the brain network boundary-aware module for prediction, to obtain a prediction probability of having a tested disease; and
an updating module, configured to reversely update parameters of the feature extraction network, the graph representation diffusion learning network, the brain network reconstruction network, and the brain network boundary-aware module based on the predicted probability and a pre-constructed loss function.

9. A computer device, comprising a processor and a memory coupled to the processor, wherein the memory stores program instructions, and when the program instructions are executed by the processor, the processor performs steps of the multi-modal brain network calculation method according to claim 1.

10. A non-transitory storage medium, wherein the non-transitory storage medium stores program instructions for implementing the multi-modal brain network calculation method according to claim 1.
